(19) United States
(12) Patent Application Publication
(10) Pub. No.: US 2002/0019014 A1
(43) Pub. Date: Feb. 14, 2002

(54) Methods of identifying prosaposin receptor agonists and antagonists

(76) Inventor: D. Elliot Parks, Del Mar, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/785,935

(22) Filed: Feb. 15, 2001

Related U.S. Application Data
(63) Continuation of application No. 08/896,181, filed on Jul. 17, 1997, now abandoned.

(51) Int. Cl. G01N 33/53; G01N 33/567

(52) U.S. Cl. 435/7.2

(57) ABSTRACT

Methods of identifying prosaposin receptor agonists and antagonists. Chemical libraries are screened with the purified receptor or transfected cells expressing the prosaposin receptor to determine which compounds bind to the receptor. Compounds which bind to the receptor are then tested using fictional assays to identify receptor agonists and antagonists.

### RELATED APPLICATIONS

This application is a continuation and claims priority to U.S. Patent Application No. 08/896,181, filed on Jul. 17, 1997, which is herein expressly incorporated by reference in its entirety.

### FIELD OF THE INVENTION

The present invention relates to the identification of compounds which bind to the prosaposin receptor and act as either agonists or antagonists thereof.

### BACKGROUND OF THE INVENTION

Prosaposin, a 70 kilodalton glycoprotein, is the precursor of a group of four heat-stable glycoproteins which are required for hydrolysis of glycosphingolipids by lysosomal hydrolases (Kishimoto et al., J Lipid Res., 33:1255-1267, 1992). Prosaposin is proteolytically processed in lysosomes to generate saposins A, B, C, and D which exist as four tandem domains in prosaposin (O'Brien et al., FASEB J, 5:301-308, 1991). All four saposins are structurally similar to each other, including the placement of six cysteines, a glycosylation site and conserved proline residues.

As described in U.S. Pat. No. 5,571,787 and International Application No. PCT/US94/08453, the entire contents of which are hereby incorporated by reference, prosaposin, saposin C and peptides derived from saposin C (18-mer and 22-mer peptides) induce neurite outgrowth, prevent neural cell death and stimulate myelination. The neurotrophic and myelinotrophic activity have been further localized to a 12-mer region (amino acids 18-29) of saposin C. Immunohistochemical studies showed that prosaposin is localized to populations of large neurons including upper and lower motor neurons. Prosaposin binds to a cell surface receptor and stimulates incorporation of ³²P into several proteins. As described in U.S. Pat. No. 5,571,787 and PCT/US94/08453, the prosaposin receptor can be isolated from brain tissue and neural cells by affinity purification of a plasma membrane P-100 fraction.

The use of peptides as therapeutic agents has inherent limitations including susceptibility to proteolysis. In the nervous system, it is desirable for therapeutic agents to cross the blood brain barrier. Although the 18-mer referred to above can cross the blood brain barrier, the identification of smaller, more stable peptides and other molecules which bind to and activate the prosaposin receptor would be beneficial in the treatment of neural and myelination disorders. The present invention provides methods for identifying such molecules.

### SUMMARY OF THE INVENTION

One embodiment of the present invention is a method of identifying a prosaposin receptor agonist, comprising the steps of contacting the prosaposin receptor with a test compound; and assaying said compound in a receptor activity assay, whereby stimulation of receptor activity indicates that the compound is a receptor agonist. Preferably, the prosaposin receptor is purified. Alternatively, the prosaposin receptor is recombinantly produced. Advantageously, the prosaposin receptor is expressed on the cell surface, coated onto an inert surface or placed on a conducting surface. The test compound may be a protein, polypeptide, peptide, peptidomimetic, small organic molecule or organonimetic. In one aspect of this preferred embodiment, the receptor activity assay is neurite outgrowth, ex vivo myelination, neural cell death or MAP kinase phosphorylation. Preferably, the test compound is contained within a chemical library. The method may further comprise the step of determining whether the compound binds to the receptor after the contacting step. Advantageously, the determining step comprises an agonist competition assay. Alternatively, the determining step comprises an antagonist competition assay.

The present invention also provides a method of identifying a prosaposin receptor antagonist, comprising the steps of contacting the prosaposin receptor with a test compound; and assaying compounds which bind to the receptor in a receptor activity assay, whereby inhibition of ligand-stimulated receptor activity indicates that the compound is a receptor antagonist. Preferably, the prosaposin receptor is purified. Alternatively, the prosaposin receptor is recombinantly produced. Advantageously, the prosaposin receptor is expressed on the cell surface. The test compound may be a protein, polypeptide, peptide, peptidomimetic, small organic molecule or organomirnmetic. In one aspect of this preferred embodiment, the receptor activity assay is neurite outgrowth, ex vivo myelination, neural cell death or MAP kinase phosphorylation. Preferably, the test compound is contained within a chemical library. The method may further comprise the step of determining whether the compound binds to the receptor after the contacting step. Advantageously, the determining step comprises an agonist competition assay. Alternatively, the determining step comprises an antagonist competition assay.

### DETAILED DESCRIPTION OF THE PREFERRED EMBODIMENTS

The present invention provides methods for identifying agonists and antagonists of the prosaposin receptor. Compounds capable of binding to the prosaposin receptor are identified, thereby identifying them as potential agonists or antagonists. These compounds may then be tested in functional assays to identify receptor agonists and antagonists. A receptor agonist is defined as a compound which has affinity for and stimulates physiologic activity at cell receptors normally stimulated by endogenous substances. In contrast, a receptor antagonist is a compound which binds to a cell receptor without eliciting a biological response and prevents the natural ligand from binding thereto. Receptor agonists both bind to the receptor and stimulate its activity. In contrast, receptor antagonists either bind to and inhibit receptor activity, or bind to and prevent binding of receptor agonists

The receptor is used to screen chemical or biological libraries of proteins, polypeptides, peptides, peptidomimetics, small organic compounds or organomimetics by well known methods. In a preferred embodiment, the screening procedure is performed using cells which constitutively express the membrane-bound prosaposin receptor. This is typically accomplished by transfecting cells, either transiently or stably, with the cDNA encoding the receptor contained within an expression vector as described below. This will allow the bioengineering, design and manipulation of signals, triggers and tags to detect both ligand binding and cell stimulation. Alternatively, the native or recombinant receptor is inserted into the cell membrane prior to library screening with whole cells containing the inserted receptor. In another embodiment, cells which endogenously express the receptor may be used. In still another embodiment, the isolated or recombinantly-produced receptor is coated onto an inert surface such as microwell plates or latex beads. The receptor may also be placed on a conducting surface (e.g. silicon wafer) prior to library screening.

The prosaposin receptor can be isolated by affinity purification as described in U.S. Pat. No. 5,571,787 using (for example) a bound 22-mer saposin C fragment. Alternatively, the receptor may be isolated using an antibody affinity column. The receptor may also be recombinantly produced by expression of a cDNA encoding the receptor in transfected prokaryotic or eukaryotic cells. In a preferred embodiment, the receptor cDNA is inserted in an expression vector and introduced into the well known baculovirus expression system.

In a preferred embodiment, the cDNA encoding the prosaposin receptor is isolated by expression cloning (Gearing et al., EMBO J, 8:3667-3676, 1989) of a neural cell cDNA library using labeled prosapsin, saposin C, or a fragment thereof including amino acids 18-29 of saposin C, capable of binding to the prosaposin receptor. The isolation of the receptor cDNA using other methods is also contemplated, including conventional cDNA library screening using an antibody generated against saposin C (polyclonal or monoclonal) and hybridization screening of cDNA libraries using degenerate oligonucleotides deduced from receptor peptide sequences. Such sequences may be obtained by Edman degradation of the purified receptor protein or by sequencing tryptic peptide fragments. These methods are well known in the art (See, for example, Sambrook et al., Molecular Cloning: A laboratory Manual, Cold Spring Harbor Laboratory Press, Cold Spring Harbor, N.Y., 1989; Ausubel et al., Current Protocols in Molecular Biology, 1989).

Once the receptor cDNA sequence is determined, mutations can be introduced to study structure-function relationships as they relate to ligand binding and effector system coupling. For example, point mutations can be introduced into the receptor by methods well known in the art. The mutant receptor can then be introduced into cells and the effect of the mutation on ligand binding and signaling pathways can be determined.

In another preferred embodiment, the cDNA encoding the receptor is placed in a eukaryotic expression vector for transient or stable transfection into a mammalian cell line. Many such cell lines are known in the art, including COS-1, COS-7 and chinese hamster ovary (CHO) cells, all of which are available from the American Type Culture Collection (ATCC) (Rockville, Md.). Many such eukaryotic expression vectors are known and commercially available. Preferred expression vectors include retroviral vectors, adenoviral vectors and SV40-based vectors. The vector may also contain a selectable marker, such as antibiotic resistance, to select for cells which are expressing the receptor. Although the preferred method of transfection is electroporation, other methods are also contemplated including calcium phosphate precipitation and DEAE-dextran. Positive transfectants are pooled and screened to determine which clones express the most prosaposin receptor by comparing transfected cells with untransfected cells in competitive binding experiments using radiolabeled prosaposin, saposin C or peptide derived therefrom in the presence of excess unlabeled prosaposin, saposin C or peptide derived therefrom. Stable transfectants are used to screen large libraries of synthetic or natural compounds, particularly phagemid or combinatorial libraries.

Peptide, nucleic acid and polysaccharide-based compounds may be synthesized by random and directed synthesis (Lam et al., Nature, 354:82-86, 1991). Alternatively, libraries of natural compounds in the form of bacterial, fungal, plant and animal extracts are available or readily producible. Additionally, natural and synthetically produced libraries and compounds are readily modified through conventional chemical, physical and biochemical means. Any desired compound may be screened for binding to the prosaposin receptor.

Peptide libraries may be synthesized in arrays on glass chips (Fodor et al., Science, 251:767-773, 1991), on plastic pins (Geysen et al., Proc. Natl. Acad. Sci. U.S.A., 81:3998-4002, 1984) or in tea-bags (Houghten, Proc. Natl. Acad. Sci. U.S.A., 82:5131-5135). Phagemid libraries expressing random peptides are prepared by inserting random oligonucleotides in frame and amino-terminal to the pVIII gene of bacteriophage f1, which encodes the major coat protein, permitting the display of peptides on the surface of the phage particles (Li et al., Nature Biotechnol., 14:986-991, 1996). Libraries are screened with either whole cells expressing the prosaposin receptor prepared as described above or with affinity purified prosaposin receptor prepared from plasma membrane P-100 fractions as described in U.S. Pat. No. 5,571,787, or in a baculovirus expression system. Phagemid library construction and screening is described in detail by Li et al., supra and by Koivunen et al., (Biotechnology, 13:265-270, 1995)

Phagemids which bind to the receptor are then analyzed to determine the sequence of the encoded peptide. To directly demonstrate the ability of the peptide to bind to the prosaposin receptor, binding assays are performed on transfected cells expressing the prosaposin receptor, on cells which endogenously express the receptor or on affinity purified receptor preparations. One such assay is a competitive binding assay in which labeled prosaposin, saposin C or a fragment thereof is incubated with the receptor in the presence of increasing concentrations of the corresponding unlabeled polypeptide. The decrease in binding of labeled polypeptide in the presence of increasing concentrations of unlabeled peptide indicates that the polypeptide binds to the receptor.

Binding assays using the expressed protein, either in whole transfected cells or in purified membrane preparations, are particularly useful in identifying compounds which bind to the prosaposin receptor. In a preferred embodiment of the invention, transfected cells expressing the prosaposin receptor or purified membrane preparations prepared as described in U.S. Pat. No. 5,571,787, are incubated with labeled prosaposin, saposin C or peptide including amino acids 18-29 of saposin C in the presence of increasing concentrations of a test compound to determine whether the test compound inhibits binding of the labeled polypeptide to the receptor. Alternatively, an agonist competition assay is performed in which prosaposin, saposin C or a saposin C-derived peptide is labeled and incubated with purified receptor or receptor-expressing cells. Drug candidates are then added and their ability to dislodge the peptide is assayed by determining how much of the labelled peptide is still bound to the receptor. These compounds are then tested for functional activity.

In perhaps the simplest version of an assay of the present invention, a compound to be tested is incubated with the receptor. After incubation, one ascertains whether the test compound has bound to the receptor. If so, the compound is a potential agonist or antagonist. Then, if desired, further assays, including fictional assays for neurite outgrowth or other evidence of neurotrophic or effector activity, can be performed to assess the type of function possessed by the test compound. Such functional assays are described, for example, in U.S. Pat. No. 5,571,787 and in Example 6 below. Actual binding can be determined through use of competitive techniques, as described elsewhere in this document. Alternatively, binding between the receptor and the test compound can be determined by using immobilized test compound and labeled receptor or immobilized receptor and labeled test compound, in each case using a separation (e.g. washing) step after incubation followed by detection of bound label. Alternatively, simple and well known molecular weight determination techniques, such as any of the well known chromatographic techniques, can be used to determine whether species of different molecular weight from the starting materials were formed in the incubation step.

Compounds which bind to the receptor may also be identified using an antagonist competition assay. In one version of this assay, a labeled inactive compound is incubated with the purified receptor or receptor-expressing cells. This compound (e.g. saposin A) binds to the receptor without stimulating any of its effector functions. A drug candidate is then added and a functional assay is performed to determine whether the compound is an agonist or antagonist as described below. The assay may further include measuring displacement of the labeled inactive compound to determine whether the drug candidate binds to the receptor prior to performing the functional assay.

Once peptides which bind to the prosaposin receptor are identified, it can then be determined whether these compounds stimulate or inhibit receptor-mediated effector functions. Compounds which bind to the prosaposin receptor are tested in functional assays to determine if they are receptor agonists or antagonists. Responses can be measured in cells expressing the receptor using signal transduction systems including, but not limited to, protein phosphorylation, adenylate cyclase activity, phosphoinositide hydrolysis, guanylate cyclase activity, ion fluxes (e.g. calcium) or pH changes. These types of responses can either be present in the host cell or introduced into the host cell along with the receptor. Four functional assays performed in receptor-containing cells are described below. One such assay is an in vitro neurite outgrowth assay which is described in Example 6. Another assay is the ability of the peptide to stimulate ex vivo myelination in mouse cerebellar explants (Example 7). A third assay is the ability of the peptide to prevent neural cell death (Example 8). Another assay is the stimulation MAP kinase phosphorylation as described in Example 9. Compounds which bind to the prosaposin receptor may also act as receptor antagonists. These compounds will bind to the prosaposin binding site of the receptor and prevent prosaposin-mediated receptor activation. To determine whether a compound is a prosaposin receptor antagonist, the functional assays described above are performed in the presence of both the compound and prosaposin, saposin C or a peptide derived from saposin C. Cells are pre-incubated with the compound to allow binding to the receptor, followed by incubation with prosaposin, saposin C or a saposin C-derived peptide. Alternatively, cells are pre-incubated with prosaposin, saposin C or a saposin C-derived peptide, followed by incubation with the compound. If neurite outgrowth, myelination or MAP kinase phosphorylation does not occur in the presence of the compound, the compound is a prosaposin receptor agonist. The use of other functional assays for identifying prosaposin receptor agonists and antagonists is also within the scope of the invention.

The cloning of the prosaposin receptor cDNA is described below.

### EXAMPLE 1

### Cloning of prosaposin receptor cDNA

A cDNA library was constructed from immortalized human Schwann cells (iSC) using a kit (Stratagene, La Jolla, Calif.). The cDNA library is screened essentially as described by Gearing et al. (EMBO J, 8:3667-3676, 1989). 1×10⁷ clones, produced by transforming E. coli cells, are sorted into 500 pools of about 2×10⁴ clones and glycerol stocks are prepared. Miniprep plasmid DNA from each pool is transfected into COS-7 cells by electroporation. Briefly, 1.5×10⁶ COS-7 cells in 180 μl phosphate-buffered saline (PBS) (pH 7.3) are mixed with 20 μl DNA (about 3 pg) and chilled on ice for 5 minutes. Cells are electroporated in 0.4 cm gap cuvettes at 300 V and 125 μFD (r=8.2−10.5 ms), returned to ice for 5 min and cultured in 2 ml Dulbecco's modified Eagle's medium (DMEM) containing 10 % fetal calf serum (FCS). Human prosaposin is radioiodinated by well known methods, for example by using chloramine-T. After 48 hours, the medium is removed and the transfected monolayers are tested for binding of radioiodinated human prosaposin in 1 ml of 10 mM HEPES-buffered RPMI medium, pH 7.2, containing 10% FCS. for 1 hour at 20° C. The monolayers are washed twice in medium, fixed in 2.5% glutaraldehyde/PBS and dipped in 1% gelatin. The slides are dipped in photographic emulsion at 42° C. and exposed in the dark for 48 hours at 4° C. in light proof boxes containing drierite. Slides are developed for 3 minutes, rinsed in water and fixed before staining. Slides are screened at 10-20× magnification. Glycerol stocks of E. coli transfornants containing radioiodinated prosaposin and thus corresponding to positive pools are partitioned into smaller pools as above until single positive cDNA clones are obtained. Conventional sequencing techniques are used to sequence the cDNA.

### EXAMPLE 2

### Transfection of cells with prosaposin receptor cDNA

Once the prosaposin receptor cDNA is identified and sequenced, it is placed into an expression vector and used to stably transfect COS-7 cells, which do not express the prosaposin receptor, using methods well known in the art. About 1×10⁴ COS-7 cells are transfected by electroporation with the prosaposin DNA contained within the pcDNA1 expression vector (Matthew et al., Cell, 85:973-982, 1991) containing a selectable marker (neomycin resistance). Cells which contain the prosaposin receptor are identified by growth in the presence of neomycin.

### EXAMPLE 3

### Radioreceptor assay

To identify compounds which bind to the prosaposin receptor, a radioreceptor assay is performed. COS-7 Cells stably expressing the prosaposin receptor are plated in six-well dishes and allowed to grow overnight. Untransfected COS-7 cells are used as a control. The cells are washed twice with 10% dimethyl sulfoxide in HEPES-buffered saline (HBS), 0.1% bovine serum albumin (BSA) and incubated at 22° C. for 90 minutes in 0.5 ml of HBS, 0.1% BSA containing 100,000 cpm of iodinated prosaposin and varying amounts of unlabeled competitor compounds. Following binding, the cells are washed three times with cold HBS, solubilized in 0.5 ml of 0.5 N NaOH and removed from the dish. Radioactivity is measured in a gamma counter. Data are expressed as percent specific binding, where 100% specific binding is the difference between binding in the absence of competitor and binding in the presence of a 100-fold molar excess of unlabeled prosaposin. The displacement of iodinated prosaposin binding to the receptor indicates that the particular compound binds to the receptor. The compounds of interest are then tested in one or more of the functional assays described in Examples 6-10 to determine whether they are receptor agonists or antagonists.

Although the preferred method of identifying prosaposin receptor ligands is through radiolabeling, other methods known in the art are also within the scope of the present invention. For instance, well known methods exist for colorimetrically and fluorimetrically labeling compounds. Reagents that are commercially available for this purpose include colored dyes and fluorescein, respectively, available from Sigma (St. Louis, Mo.) and Molecular Probes (Junction City, Oreg.).

The affinity purified prosaposin receptor or receptor purified from a baculovirus expression system is used to screen random peptide libraries contained within phagemid vectors as described in the following example.

### EXAMPLE 4

### Construction of phagemid peptide libraries

Peptide libraries are constructed as described by Koivunen et al. (Bio/Technology, 13:265-270, 1995). Peptide libraries are constructed on fuse 5 vector. The CX₅C, CX₆C and CX₉ libraries are prepared using synthetic oligonucleotides containing core sequences TGT(NNK)₅TGT (SEQ ID NO: 1); TGT(NNK)₆TGT (SEQ ID NO: 2); and TGT(NNK)₉ (SEQ ID NO: 3), where N is an equimolar mixture of A, T, G and C; K=G or T. The oligonucleotides were made double-stranded by PCR amplification with three cycles, purified and ligated to the N-tenninus of the pIII gene of fuse vector. The CX₅C, CX₆C and CX₉ vectors were transfected into MC 1061 cells using 16, 60 and 250 electroporations, respectively. Bacteria were cultured for 24 hours in the presence of 20 μg/ml tetracycline, and phage were collected from the supernatant by precipitation twice with polyethylene glycol. The phage pellets are dissolved in approximately 10¹³ transducing units/ml in TBS buffer containing 0.02% sodium azide and stored at 4° C.

### EXAMPLE 5

### Screening of phagemid peptide library

A mixture of phage containing 7×10^{10, 2.5×10}¹¹, 2.5×10¹¹ and 4×10¹¹ transducing units from each of the CX₅C, CX₆C, CX₇C and CX₉ libraries, respectively, are screened with affinity-purified prosaposin receptor, or COS-7 cells expressing the prosaposin receptor, coated on microtiter wells as described (Koivunen et al., J Biol. Chem., 268:20205-20210, 1993). In the first panning, receptor is coated at 5 μg/well, and the library pool is incubated for 4 hours at 25 ° C. in TBS buffer containing 1% BSA. Phage remaining bound after extensive washing are eluted with a glycine buffer at pH 2.2. Any tight-binding phage possibly remaining bound to the wells are captured by incubating with concentrated K91kan bacteria for 2 hours at 37° C. Bacteria are mixed with the low pH eluate and the phage are amplified. In the subsequent pannings, the receptor is coated at lower concentrations (100, 10 and 1 ng/well) to select high affinity phage sequences and phage were randomly selected from plates with 20-fold or higher enrichment of phage relative to the background.

### EXAMPLE 6

### Neurite outgrowth assay

NS20Y neuroblastoma cells are grown in DMEM containing 10% FCS and 1 mM sodium pyruvate. Cells are removed with trypsin and plated in 30 mm petri dishes onto glass coverslips. After 20-24 hours, the medium is replaced with DMEM containing 0.5% FCS plus the compound of interest at a starting concentration of from about 0.1 mm to about 10 mM. Prosaposin, saposin C or an active neurotrophic fragment thereof is used as a positive control at a concentration of between about 0.1 μg/ml and 5 μg/ml. Cells are cultured for another 24 hours, washed with PBS and fixed with Bouin's solution for 30 minutes. Neurite outgrowth is scored under a phase contrast microscope. Cells exhibiting one or more clearly defined neurites equal to or longer than one cell diameter are scored as positive. At least 200 cells are scored in different portions of each dish to determine the percentage of neurite bearing cells and assays are performed in duplicate. An increase in neurite outgrowth in response to a compound compared to untreated cells indicates that the compound is a receptor agonist.

To determine if a compound is a receptor antagonist, cells are incubated with the compound prior to addition of prosaposin, saposin C or an active neurotrophic fragment thereof. Positive control cells are not preincubated with the compound. A decrease in neurite outgrowth in cells preincubated with the compound indicates that the compound is a receptor antagonist.

### EXAMPLE 7A

### Ex vivo Myelination assay

Newborn mouse cerebellar explants are prepared according to Satomi (Zool. Sci., 9:127-137, 1992). Neurite outgrowth and myelination are observed for 22 days in culture, during the period when the newborn mouse cerebellum normally undergoes neuronal differentiation and myelination begins. Compounds of interest are added on the second day after preparation of the explants (three control and three treated explants) and outgrowth of neurites and myelination are assessed under a bright field microscope with a video camera. Prosaposin, saposin C or an active neurotrophic fragment thereon containing amino acids 18-29 of saposin C is used as a positive control at concentrations of about 1 μg/ml to 10 μg/ml. Receptor agonists promote a similar extent of neurite outgrowth and myelination compared to positive controls. To determine if a compound is a receptor antagonist, preincubation with the compound is performed as described in the previous example.

Alternatively, mylelination may be assayed by incorporation of ³⁵S into sulfolipids as described below.

### EXAMPLE 7B

### Incorporation of ³⁵S into sulfolipids

Sulfolipids are incorporated exclusively into myelin. Primary myelin-containing Schwann cells are incubated in low sulfate media (DMEM) containing 0.5% fetal bovine serum (FBS), followed by addition of ³⁵S-methionine for 48 hours. Cells are rinsed with PBS, harvested and sonicated in 100 μl distilled water. An aliquot of cell lysate is removed for protein analysis and the remainder is extracted with 5 ml chloroform/methanol, 2:1 (v/v). Schwann cell lipid extracts are chromatographed and immunostained with an anti-sulfatide monoclonal antibody as described (Hiraiwa et al., Proc. Natl. Acad. Sci. U.S.A. 94:4778-4781).

### EXAMPLE 8

### Prevention of neuronal cell death

Prosaposin, saposin C and fragments of saposin C containing amino acids 18-29 thereof also prevent neuronal cell death. NS20Y cells are plated as described in Example 6 and grown on glass coverslips in 0.5% FBS for two days in the presence or absence of a compound capable of binding to the prosaposin receptor at a starting concentration of from about 0.1 mm to about 10 mM. Prosaposin, saposin C or a fragment of saposin C is used as a positive control at a concentration of between about 0.1 μg/ml and 5 μg/ml. Media is removed and 0.2% trypan blue in PBS is added to each well. Blue-staining dead cells are scored as a percentage of the total on an inverted microscope, counting 400 cells in four areas of each well. The average error of duplicates is ±5%. A significant reduction of the number of trypan blue-positive (dead) cells indicates that the compound is a receptor agonist and can rescue neural cells from programmed cell death.

### EXAMPLE 9

### MAP kinase (MAPK) phosphorylation assay

Prosaposin, saposin C and active neurotrophic fragments thereof stimulate phosphorylation of MAPK after binding to the prosaposin receptor. PC12 cells are grown to 85% confluence in DME high glucose media containing 10% FBS, 5% horse serum, 100 U/ml penicillin, 100 μg/ml streptomycin and 250 pg/ml fimgizone. Approximately 2×10⁶ cells per 100 mm plate are placed in serum-free media for 24 hours prior to addition of compounds capable of binding to the prosaposin receptor. Positive controls are as described previously. Cells are stimulated with the compound of interest, then lysed on ice in 1 ml of 50 mM Tris-HCl, pH 8.0, 150 mM NaCl, 1 mM EGTA, pH 8.0, 100 mM NaF, pH 8.0, 10% glycerol, 1.5 mM MgCl₂. 1% (v/v) Triton X-100, 1 mM sodium orthovanadate, 1 mM phenylmethylsulfonyl fluoride (PMSF), 10 μg/ml leupeptin, 20 μg/ml aprotinin. Unsolubilized materials are removed by ultracentrifugation at 12,000 x g at4° C.

Equal amounts of cell lysates are resolved by sodium dodecyl sulfate-polyacrylamide gel electrophoresis (SDS-PAGE) and transferred to a PVDF membrane (Millipore, Bedford, Mass.). Phosphorylated MAPK is identified using PhosphoPlus™ MAPK antibody and an alkaline phosphatase conjugated secondary antibody that emits light in the presence of a chemiluminescent substrate (New England Biolabs, Beverly, Mass.). After phosphorylated MAPK proteins are identified, blots are stripped in 62.5 mM Tris-HCI, pH 6.8, 2% SDS, 100 mM β-mercaptoethanol at 50° C. for 30 minutes, washed and incubated with the MAPK-C antibody. The blots are developed as described above. Autoradiographs are scanned using an IrnageQuant™ (Molecular Dynamics, Sunnyvale, Calif.). Densitometric data are expressed as a ratio of MAPK-P absorbance units divided by MAPK-C absorbance units. The higher this number, the higher the phosphorylation of MAPK.

It should be noted that the present invention is not limited to only those embodiments described in the Detailed Description. Any embodiment which retains the spirit of the present invention should be considered to be within its scope. However, the invention is only limited by the scope of the following claims.

What is claimed is:

1. A method of identifying a compound that binds to a prosaposin receptor comprising:
(a) incubating a first test compound with said receptor; and
(b) determining whether said first test compound binds to said receptor.

2. The method of claim 1, wherein said receptor is isolated by passage of a solubilized neural plasma membrane P-100 fraction through an affinity column comprising amino acids 8-29 of saposin C.

3. The method of claim 1, wherein said receptor is coated onto a surface or is placed on a conducting surface.

4. The method of claim 1, wherein said first test compound is a protein, polypeptide, peptide, peptidomimetic, small organic molecule or organomimetic.

5. The method of claim 1, wherein said first test compound is contained within a library of proteins, polypeptides, peptides, peptidomimetics, small organic compounds or organomimetics.

6. The method of claim 1, wherein said determining step comprises a competition assay.

7. The method of claim 1, further comprising the steps of repeating steps (a) and (b) with a second test compound.

8. The method of claim 1, further comprising the step of performing a functional assay to ascertain whether said first test compound identified in steps (a) and (b) has agonist or antagonist function.


SEQUENCE LISTING (3 sequences)

SEQ ID NO: 1 - DNA (cDNA), 18 bp; single-stranded, linear
tgtnnknnknnknnknnk

SEQ ID NO: 2 - DNA (cDNA), 24 bp; single-stranded, linear
tgtnnknnknnknnknnknnktgt

SEQ ID NO: 3 - DNA (cDNA), 30 bp; single-stranded, linear
tgtnnknnknnknnknnknnknnknnknnk